(19) United States
(12) Patent Application Publication
(10) Pub. No.: US 2017/0218035 A1
(43) Pub. Date: Aug. 3, 2017

(54) COMPOSITIONS AND METHODS FOR TREATMENT WITH HEMOPEXIN

(71) Applicant: Bayer HealthCare LLC, Whippany, NJ (US)

(72) Inventors: Kirk MCLEAN, Orinda, CA (US); Terry HERMISTON, Mill Valley, CA (US); Alan BROOKS, Clayton, CA (US); Richard FELDMAN, El Cerrito, CA (US)

(73) Assignee: Bayer HealthCare LLC, Whippany, NJ (US)

(21) Appl. No.: 15/515,304

(22) PCT Filed: Sep. 29, 2015
(86) PCT No.: PCT/US2015/052990

§ 371 Date: Mar. 29, 2017

Related U.S. Application Data
(60) Provisional application No. 62/057,613, filed on Sep. 30, 2014.

(51) Int. Cl. C07K 14/47

(52) U.S. Cl. CPC C07K 14/47; A61K 38/00

(57) ABSTRACT

Compositions and methods are provided for therapeutic treatment using recombinant Hemopexin molecules having sufficient sialyation and/or absence of neutral glycans to allow for sufficient circulation to remove free heme from a biological organism. In other embodiments, a recombinant Hemopexin molecule is provided for therapeutic treatment having a percentage of neutral glycans to total glycans in a range of from about 2 to about 30 percent as measured by HPLC after labelling with fluorescent probe 2-aminobenzoic acid. Methods of treatment and making a recombinant Hemopexin molecule are also described.

### SEQUENCE LISTING SUBMISSION

The sequence listing associated with this application is filed in electronic format via EFS-Web and hereby incorporated by reference into the specification in its entirety. The section headings used herein are for organizational purposes only and are not to be construed as limiting the subject matter described in any way.

### BACKGROUND

Heme serves a variety of functions in biological organisms. It is a critical component of hemoproteins such as cytochromes, DNA synthetic enzymes, myoglobin, and hemoglobin. However, free heme at high levels can be toxic and failure to control free heme can result in a variety of diseases and disorders.

In diseases with accelerated hemolysis such as sickle cell disease (SCD) and β-thalassemia (BThall) heme levels are elevated compared to normal controls. The elevated heme levels are caused by the release of hemoglobin from lysed red blood cells which, following mild oxidation, releases the heme moiety. Free hemoglobin and heme scavenge nitric oxide and catalyse the formation of reactive oxygen intermediates which are cytotoxic and induce pro-inflammatory responses in cells. The liver plays a crucial role in helping to regulate heme levels. The liver works in conjunction with various proteins (including FLVCR) to export excess heme to the bile and feces. In normal individuals two proteins haptoglobin and Hemopexin scavenge the free hemoglobin and heme, respectively, and thereby reduce the associated cytotoxic and pro-inflammatory effects.

Hemopexin is a plasma based glycoprotein that protects against heme mediated toxicity associated with haemolytic and infectious diseases. This protein becomes severely depleted in some clinical settings such as Sickle Cell Disease (SCD) and Thalassemia. Hemopexin has the highest known binding affinity for heme (reported to be Kd<1 pM). Furthermore, in addition to reducing the toxic effects of free heme, Hemopexin can reduce the negative effects of free hemoglobin, presumably due to its ability to scavenge associated toxic heme. In hemolytic diseases both haptoglobin and Hemopexin can become severely depleted leaving hemoglobin and heme free to exert their negative effects. Hemopoexin can also act as a heme scavenger to reduce the toxic effects of free heme in hemolytic diseases. For example human plasma derived Hemopexin has been shown to reduce cytoxic and pro-inflammatory effects of free heme and improve vascular function in SCD and Bthall mouse models. Hemopexin has been shown to bind and sequester intravascular heme and reduce its associated toxicity.

Human plasma derived Hemopexin is a fully sialylated plasma glycoprotein that has a circulating half-life of 7 days. Upon binding heme a conformational change occurs in Hemopexin that increases its affinity for the LRP receptor on hepatocytes causing a rapid removal of the complex from the circulation (T ½=7 hours). Hemopexin is extensively glycosylated with both N and O-linked carbohydrates. Proper sialylation of galactose residues on N-Linked glycans can have a significant impact on clearance properties of proteins in vivo. Insufficient sialylation can lead to more rapid clearance through the asialylglycoprotein receptor on hepatocytes removing the protein from circulation before it has a chance to deliver a therapeutic benefit. This can be especially problematic for recombinant proteins when pushing for high expression levels where glycosylation and sialylation pathways can be unable to keep up with the rate of protein production.

The bioavailability of the protein is a crucial factor impacting or alleviating certain diseases or their associated symptoms. Further, another limiting factor for therapeutic treatment use of Hemopexin appears to be the high levels of protein that need to be administered. This is likely due to the high turnover rates seen in diseases with accelerated hemolysis. While plasma derived Hemopexin could be used as a source for clinical development it has inherent risks such as potential for disease transmission (e.g. HCV, HIV) to patients. Improvements in the production process of recombinant Hemopexin can improve the likelihood that such a protein can be made using a commercially viable process.

There remains a need for effective compositions and methods for therapeutic treatment and heme removal from cells and plasma of biological organisms. Further, there is a necessity for heme export from cells and plasma to reduce the toxicity of excess heme and prevent various biological disorders associated with these imbalances.

### SUMMARY

Compositions and methods are provided for therapeutic treatment comprising recombinant Hemopexin molecules having sufficient sialyation and/or sufficiently low levels or an absence of neutral glycans to allow for sufficient circulation to remove free heme from a biological organism.

In some embodiments, a recombinant Hemopexin molecule is provided for therapeutic treatment comprising a percentage of neutral glycans to total glycans in a range of from about 2 to about 30 percent as measured by HPLC after labelling with fluorescent probe 2-aminobenzoic acid. In at least one embodiment, the recombinant Hemopexin molecule may be expressed from a CHO cell, such as a CHO-K1 cell. In at least one embodiment, the recombinant Hemopexin molecule may comprise a mammalian Hemopexin molecule.

In at least one embodiment, the recombinant Hemopexin molecule is for therapeutic treatment a comprises a percentage of neutral glycans in the range of from about 2 to about 30 percent, a percentage of mono-sialylated glycans in the range of from about 2 to about 40 percent, and a percentage of di/tri sialylated glycans in the range of from about 20 to about 90 percent, as measured by HPLC after labelling with fluorescent probe 2-aminobenzoic acid. In at least one embodiment, the Hemopexin molecule is used to treat the toxic effects of heme in a disease, such as sickle cell disease or β-thalassemia.

In at least one embodiment, the Hemopexin molecule comprises a percentage of neutral glycans to total glycans that is less than 30 percent as measured by HPLC after labelling with fluorescent probe 2-aminobenzoic acid. In at least one embodiment, the percentage of neutral glycans to total glycans is less than 20 percent, or less than 10 percent, as measured by HPLC after labelling with fluorescent probe 2-aminobenzoic acid.

In other embodiments, a recombinant Hemopexin molecule is provided having a 90% or great homology to SEQ ID NO: 1, wherein the percentage of neutral glycans to total glycans is in a range of from about 2 to about 30 percent as measured by HPLC after labelling with fluorescent probe 2-aminobenzoic acid.

Methods of making a recombinant Hemopexin molecule are also provided. In some embodiments the methods of making the recombinant Hemopexin molecules having a percent neutral glycan to total glycans in a range of from about 2 to about 30 percent as measured by HPLC after labelling with fluorescent probe 2-aminobenzoic acid, comprise inserting an appropriate insert and vector into a CHO cell; and expressing the recombinant Hemopexin molecule from the CHO cell wherein percent neutral glycan of the recombinant Hemopexin is in a range of from about 2 to about 30 percent as measured by HPLC after labelling with fluorescent probe 2-aminobenzoic acid. In at least one embodiment of the method, the CHO cell comprises a CHO-K1 cell.

In other embodiments, methods of therapeutic treatment using Hemopexin are also provided. In some embodiments, the methods of treatment comprise administering to a subject a recombinant Hemopexin molecule having a percentage neutral glycan to total glycans in a range of from about 2 to about 30 percent as measured by HPLC after labelling with fluorescent probe 2-aminobenzoic acid. In at least one embodiment, the recombinant Hemopexin molecule circulates in the blood stream at a sufficient half-life to bind free heme.

In another aspect of the disclosure, the recombinant Hemopexin molecule is used to reduce intravascular and/or intracellular heme for treating a disease selected from sickle cell disease, β-thalassemia, ischemia reperfusion, erythropoeitic protoporphyria, porphyria cutanea tarda, malaria, rheumatoid arthritis, anemia associated with inflammation, hemochromatosis, paroxysmal nocturnal hemoglobinuria (PNH), glucose-6-phosphate dehydrogenase deficiency, hemolytic uremic syndrome (HUS), thrombotic thrombocytopenic purpura (TTP), pre-eclampsia, sepsis, acute bleeding, and complications associated with transfusion with blood or blood substitutes, and organ preservation associated with transplantation.

In another aspect of the disclosure, the recombinant Hemopexin molecule is used in a method for exporting heme from a cell comprising contacting the cell with a recombinant hemopexin molecule comprising a percentage of neutral glycans to total glycans in a range of from about 2 to about 30 percent as measured by HPLC after labelling with fluorescent probe 2-aminobenzoic acid. In at least one embodiment, the recombinant Hemopexin molecule is used in a method of treating a disorder associated with free heme toxicity comprising administering to a subject in need thereof an effective amount of a recombinant hemopexin molecule comprising a percentage of neutral glycans to total glycans in a range of from about 2 to about 30 percent as measured by HPLC after labelling with fluorescent probe 2-aminobenzoic acid. Preferably, the disorder is selected from sickle cell disease, β-thalessemia, erythropoeitic protoporphyria, porphyria cutanea tarda, ischemia reperfusion, and malaria.

In at least one embodiment, the recombinant Hemopexin molecule is used in a method of treating a disorder associated with excess intravascular or intracellular heme comprising administering to a subject in need thereof an effective amount of a recombinant hemopexin molecule comprising a percentage of neutral glycans to total glycans in a range of from about 2 to about 30 percent as measured by HPLC after labelling with fluorescent probe 2-aminobenzoic acid. Preferably, the disorder is selected from sickle cell disease, β-thalessemia, rheumatoid arthritis, anemia associated with inflammation, and other conditions in which heme accumulates in cells.

These and other features of the present teachings are set forth herein.

### BRIEF DESCRIPTION OF THE DRAWINGS

The skilled artisan will understand that the drawings, described below, are for illustration purposes only. The drawings are not intended to limit the scope of the present teachings or claims in any way.

FIG. 1 shows expression of recombinant human Hemopexin in selected high expressing CHOK1 and CHO-S clones. Expression levels were determined via an anti-human Hemopexin ELISA kit.

FIG. 2 shows a determination of EC50's for inhibition of a heme dependent peroxidase assay using conditioned media from various high expressing CHOK1 and CHO-S derived Hemopexins. Assays were performed using a commercially available heme dependent peroxidase assay.

FIG. 3 shows a flow chart summarizing glycan analysis.

FIG. 4 shows sialylated N-Glycan MALDI analysis for a subset of clones from screening.

FIG. 5 shows neutral N-Glycan MALDI analysis for a subset of clones from screening.

FIG. 6 shows % Neutral glycans based on 2AA analysis for various CHOK1 and the CHOS clones.

FIG. 7 graph showed % neutral glycans for CHOK1 clones vs. CHO-S derived hemopexin

FIG. 8 shows a plot of % Neutral glycan versus expression levels for CHOK1 clones. Selected clones are circled. CHOK1-76 clone is indicated with an arrow.

FIG. 9 shows neutral N-glycan MALDI analysis of plasma derived (pd-HPX), two batches of CHOK1 clone 76 (CHOK1 batches A and B), and CHOS derived Hemopexin used for pharmacokinetic analysis.

FIG. 10 shows sialylated N-glycan MALDI analysis of plasma derived (pd-HPX), two batches of CHOK1 clone 76 (CHOK1 batch A and batch B), and CHOS derived Hemopexin used for pharmacokinetic analysis.

FIG. 11 shows 2AA Analysis showing % neutral glycans for plasma derived (pd-HPX), two batches of CHOK1 clone 76 (CHOK1 batch A and batch B), and CHOS derived Hemopexin used for pharmacokinetic analysis.

FIG. 12 shows 2AA Analysis showing % neutral, monosialylated and di and trisialylated N-glycans in CHOK1 clone 76 derived hemopexin purified protein derived from bioreactor cultures harvested on day 7, 11, and 14.

FIG. 13 shows a pharmacokinetic analysis of recombinant (r-HPX) and plasma derived Hemopexin (pd-HPX) in Sprague-Dawley rats.

### DETAILED DESCRIPTION

This disclosure provides compositions and methods for treatment with Hemopexin and/or recombinant Hemopexin. The compositions and methods can be administered to a subject having one or more diseases or symptoms. In certain instances the diseases can be associated with elevated levels of heme.

For the purpose of interpreting this specification, the following definitions will apply. In the event that any definition set forth below conflicts with the usage of that word in any other document, including any document incorporated herein by reference, the definition set forth below shall always control for purposes of interpreting this specification and its associated claims unless a contrary meaning is clearly intended (for example in the document where the term is originally used).

Whenever appropriate, terms used in the singular will also include the plural and vice versa. The use of “a” herein means “one or more” unless stated otherwise or where the use of “one or more” is clearly inappropriate. The use of “or” means “and/or” unless stated otherwise. The use of “comprise,” “comprises,” “comprising,” “include,” “includes,” and “including” are interchangeable and are not limiting. The term “such as” also is not intended to be limiting. For example, the term “including” shall mean “including, but not limited to.”

As used herein, the term “about” refers to +/−10% of the unit value provided. As used herein, the term “substantially” refers to the qualitative condition of exhibiting a total or approximate degree of a characteristic or property of interest. One of ordinary skill in the biological arts will understand that biological and chemical phenomena rarely, if ever, achieve or avoid an absolute result because of the many variables that affect testing, production, and storage of biological and chemical compositions and materials, and because of the inherent error in the instruments and equipment used in the testing, production, and storage of biological and chemical compositions and materials. The term substantially is, therefore, used herein to capture the potential lack of completeness inherent in many biological and chemical phenomena.

The term “Hemopexin” or “plasma derived Hemopexin” or “HPx” or “pd-HPX” as used herein refers to any variant, isoform, and/or species homolog of Hemopexin in its form that is naturally expressed by cells and present in plasma and is distinct from recombinant Hemopexin.

The term “ recombinant Hemopexin” or “rHPx” as used herein refers to any variant, isoform, and/or species homolog of Hemopexin in its form that is expressed from cells and is distinct from plasma derived Hemopexin.

The term “therapeutically effective amount” means an amount of Hemopexin or protein combination that is needed to effectively remove excess heme in vivo or otherwise cause a measurable benefit in vivo to a subject in need thereof. The precise amount will depend upon numerous factors, including, but not limited to the components and physical characteristics of the therapeutic composition, intended patient population, individual patient considerations, and the like, and can readily be determined by one skilled in the art.

A number of factors have limited the ability to use Hemopexin as a molecule or composition for therapeutic treatment.

A first factor limiting the use of Hemopexin for therapeutic treatments is the high levels of protein that need to be administered. This is likely due to the high turnover rate seen in diseases with accelerated hemolysis. Existing methods obtain Hemopexin through extracting, purifying, and concentrating the protein from plasma. This is a time consuming and extensive process that yields limited protein.

A second factor limiting the use of plasma derived Hemopexin concerns the possible issues created by disease transmission. For instance, while plasma derived Hemopexin could be used as a source for clinical development these compositions have inherent risks such as potential for disease transmission (e.g. HCV, HIV) to patients. Further, plasma derived samples and compositions include the possibility of having various viruses and bacteria that cause disease. There is a potential risk that these pathogens are not removed and/or filtered prior to scale up production.

A third factor limiting the use of Hemopexin as a therapeutic concerns the inability to both express the protein at a high level in an efficient production process while retaining the inherent properties necessary for the protein to function and/or operate similar to the in vivo or naturally occurring proteins. Free plasma derived hemopexin has been reported to have a plasma half-life of 7 days. Upon binding of heme, the conformation of hemopexin changes, increasing its affinity for LRP on hepatocytes leading to more rapid removal from the circulation (T½=7 hours). Plasma derived hemopexin is extensively glycosylated containing 5 N-linked glycosylation sites and 1 or O-linked glycosylation sites. For plasma derived hemopexin, the N-linked carbohydrates are fully sialylated on terminal galactose carbohydrates preventing recognition and removal by the asialylglycoprotein receptor (ASGPR) in the liver. Incomplete sialylation of terminal galactose sugars on N-linked carbohydrates in recombinantly produced hemopexin would be expected to yield a protein that is much more rapidly cleared from the circulation. Since clearance of improperly sialylated hemopexin through ASGPR would occur more rapidly, independent of heme binding, this would be expected to lead to a hemopexin molecule that has reduced therapeutic potency. The percent neutral N-glycans (based on total N-glycans) determined by 2AA analysis is inversely correlated to the degree of sialylation and therefore compositions with reduced percent neutral glycan have increased levels of sialylation. In this application, we describe an expression system that produces sufficiently sialylated hemopexin at high levels, demonstrate the negative effects of under sialylation on clearance properties, and show that hemopexins with percent neutral N-glycans below 30%, below 25%, below 20%, below 15%, and below 10% will be the most useful compositions for treatment of patients. The present compositions and methods, therefore, provide unexpected benefits not obtained by pd-HPX molecules and other compositions.

For instance, improvements in the production process of recombinant Hemopexin can improve the likelihood that such a protein can be made using a commercially viable process. However, use of a general expression system results in inadequate sialylation of the Hemopexin molecules or compositions. Further, it is, therefore, desirable to decrease the levels of neutral glycans present in the molecule relative to the total glycans to improve overall composition of the Hemopexin molecules and the circulation times in vivo. Molecules and/or compositions that are neutral in charge are contacted and removed by the liver. Hence, their circulation time in the blood stream would be shorter and their clearance would be less driven by formation of a complex with free heme. Further, it should also be noted that the therapeutic molecules or compositions must have similar enough characteristics to the plasma derived or wild type Hemopexin to tightly bind free heme in the blood stream. The present compositions and methods, therefore, provide unexpected benefits not obtained by pd-HPX molecules and compositions.

Proper sialyltion of galactose residues on N-Linked glycans, therefore, can have a significant impact on clearance properties of proteins in vivo. Insufficient sialylation can lead to more rapid clearance through the asialylglycoprotein receptor on hepatocytes. This can be especially problematic for recombinant proteins when pushing for high expression levels. Both naturally occurring and recombinant Hemopexin are extensively glycosylated with both N- and O-linked carbohydrates. The percent neutral glycans can be determined using analytical methods such as 2AA analysis. The percent neutral N-glycans determined as such will be inversely proportional to the degree of sialylation. Glycan structures presenting more than one unsialylated galactose on a single carbohydrate chain would be expected to have the highest affinity for ASGPR and be cleared most rapidly.

In the production of recombinant Hemopexin, cells that produce insufficiently sialylated material lead to a rapidly cleared form of Hemopexin. In contrast we have shown that when material is expressed in cells that produce material with a greater degree of siaylylation reduced clearance rates are observed. Expression in cells that produce sufficiently sialylated material coupled with a purification process that produces recombinant Hemopexin with percent neutral N-glycans below 30%, below 25%, below 20%, below 15%, and below 10% will be more useful for treatment of patients.

Various methods can be employed to further reduce the level of neutral glycans in a Hemopexin molecule or composition and increase the levels of sialylation. These methods comprise using various defined cell lines, improving the media feed with particular excipients or nutrients, using inhibitors including but not limited to metals or their derivatives to block the sialidase enzymes that remove sialic acid from N-glycans, and implementing mutations into the polypeptide sequence to engineer in or out various amino acids to influence N-glycosylation patterns and degree of sialylation. We have shown that use of cell lines with increased propensity to add sialic acid to N-glycans can be used and the selection of clones from within a population of tranfected cells that have an increased propensity to add sialic acid to N-glycans can be used. Modification of cells with DNA coding for proteins known to influence sialylation processes to include but not be limited to sialic acid transporters, sialyltransferases, sialidase inhibitors, or siRNA and equivalent technologies.

Replenishment therapy using a recombinant produced protein is challenging, at least in part, due to the high levels of protein needed. Furthermore, hemopexin has extensive post translational modifications that combined with proper folding may differentially impact and influence individual properties of this protein. The invention herein relates to the generation and use of Hemopexin and/or recombinant Hemopexin to treat diseases.

The recombinant Hemopexin molecule may have a sequence that has 90% or greater homology to SEQ ID NO: 1. The deviations in the sequence may be caused by factors such as deletion, addition, substitution, or insertion, whether naturally occurring or introduced by directed mutagenesis or other synthetic or recombinant techniques. Furthermore, homology means that there is a functional and/or structural equivalence between the respective nucleic acid molecules or the proteins encoded therefrom. In at least one embodiment, nucleic acid molecules that are homologous to SEQ ID NO: 1 have the same biological functions as SEQ ID NO: 1.

### Pharmaceutical Uses

Hemopexin can be used for therapeutic purposes for treating genetic and acquired deficiencies or defects in heme regulation. For example, the proteins in the embodiments described above can be used to remove excess heme from the blood or plasma.

Hemopexin has therapeutic use in the treatment of disorders of heme, including disorders involving excess free vascular heme and disorders involving excess intracellular heme. Free heme toxicity disorders include sickle cell disease, β-thalassemia, ischemia reperfusion, erythropoeitic protoporphyria, porphyria cutanea tarda, and malaria. Excess free heme can lead to organ, tissue, and cellular injury or dysfunction by catalyzing the formation of reactive oxygen species. Disorders associated with excess intracellular heme include rheumatoid arthritis, anemia associated with inflammation, and other conditions in which iron accumulates in macrophage cells and cannot be recycled to red blood cells. Other diseases with excess iron/iron overload that could benefit from therapeutic use of hemopexin include hemochromatosis, paroxysmal nocturnal hemoglobinuria (PNH), glucose-6-phosphate dehydrogenase deficiency or a secondary phenomenon (e.g. hemolytic uremic syndrome (HUS), thrombotic thrombocytopenic purpura (TTP), pre-eclampsia, malaria, sepsis, and other infectious and/or inflammatory diseases, acute bleeding, and complications associated with transfusion with blood or blood substitutes, and organ preservation associated with transplantation. There would be potential benefit in any disease in which there is extensive cell lysis particularly red blood cell lysis. Diseases associated with extensive breakdown of muscle that liberate high amounts of myoglobin may also benefit from hemopexin administration.

Such disorders can be treated by administering a therapeutically effective amount of the Hemopexin to a subject in need thereof. The Hemopexin molecules and compositions also have therapeutic use in the treatment of rare diseases like SCD. Thus, also provided are methods for treating SCD and other related diseases.

The Hemopexin may be formulated for parenteral administration (e.g. by injection, for example bolus injection or continuous infusion) and may be presented in unit dose form in ampules, pre-filled syringes, small volume infusion or in multi-dose containers with an added preservative. The compositions may take such forms as suspensions or solutions, and may contain formulatory agents such as suspending, stabilizing and/or dispersing agents. As used herein, the term “parenteral” includes subcutaneous, intravenous, intramuscular, intra-articular, intra-synovial, intrasternal, intrathecal, intrahepatic, intralesional, and intracranial administration.

The Hemopexin proteins can be used as monotherapy or in combination with other therapies to address a heme disorder. The pharmaceutical compositions can be parenterally administered to subjects suffering from heme deficiency at a dosage and frequency that can vary with the severity of the disease, or, in the case of prophylactic therapy, can vary with the severity of the iron deficiency.

The compositions can be administered to patients in need as a bolus or by continuous or intermittent infusion. For example, a bolus administration of Hemopexin proteins can typically be administered by infusion extending for a period of thirty minutes to three hours. The frequency of the administration would depend upon the severity of the condition. Frequency could range from once or twice a day to once every two weeks to six months. Additionally, the compositions can be administered to patients via subcutaneous injection. For example, a dose of 1 to 8000 mg of hemopexin can be administered to patients via subcutaneous injection daily, weekly, biweekly or monthly.

### EXAMPLE 1

### Expression, Purification, and Analysis of Recombinant Hemopexin

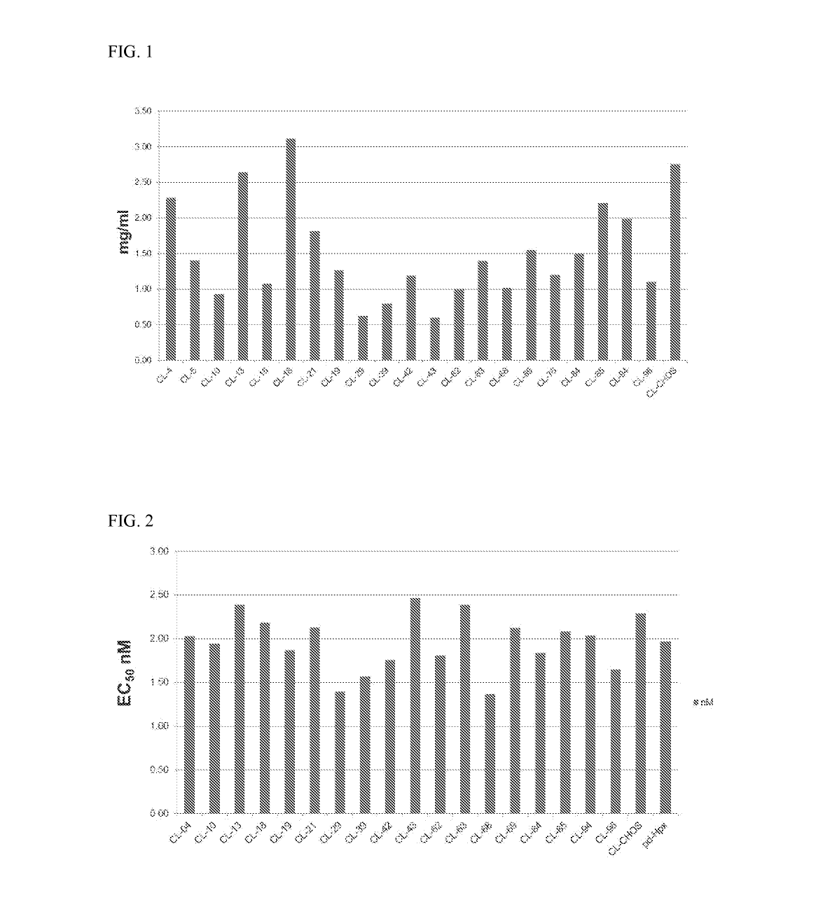

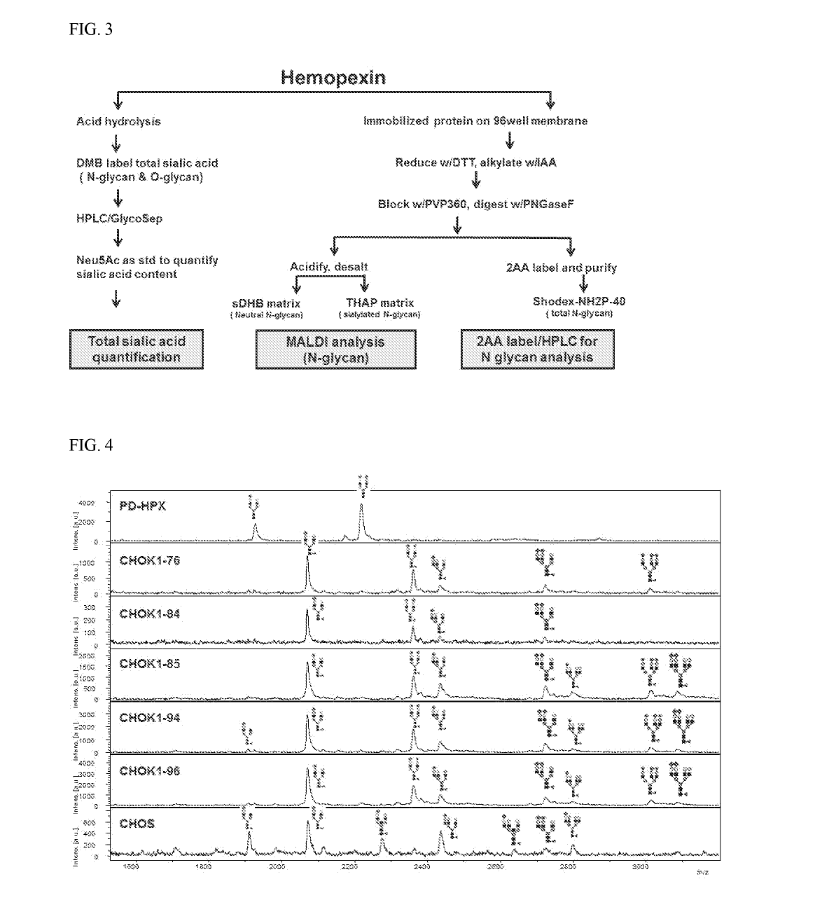

High level expression was demonstrated in CHO cells using the DNA 2.0 optimized Hemopexin cDNA sequence (SEQ ID NO: 3) with the native Hemopexin signal sequence. A similar process for identification of high expressing clones was used for both CHOS and CHOK1 cells. The process used for the CHOK1 clones was as follows: CHOK1 cells were transfected with the expression vector containing the codon optimized Hemopexin cDNA. A total of 300 CHOK1 clones were selected by limited dilution cloning in 96 well plates. Conditioned media from the clones were assayed using commercially available Hemopexin ELISA kit (ALPCO, 41-HMPHU-E01). From the original 300 clones 21 high producers were identified and subsequently evaluated using a small scale fed batch expression process (50 ml) using ActiCHO media from GE Healthcare Life Sciences. The Hemopexin protein was purified (to >95% purity) using a two-step process that included ion exchange chromatography (Q-Sepharose, GE Healthcare Life Sciences) or metal chelate chromatography (Ni-IMAC) followed by size exclusion chromatography (SD200, GE Healthcare Life Sciences). The purified proteins were evaluated for purity using SDS PAGE (4-12% Bis Tris gels) and analytical size exclusion chromatography (SD200, 10/300). Purified samples were also analyzed for heme binding using a competitive heme binding assay (based on a heme dependent peroxidase) and then submitted for glycan analysis (methods outlined below). Similar maximum protein expression levels were achieved in both CHOK1 and CHO-S cell lines (FIG. 1). The EC50's for inhibition of a heme dependent peroxidase assay was determined using a commercially available kit. All clones inhibited heme dependent peroxidase activity with a similar potency (FIG. 2). Purified proteins were submitted for glycan analysis. Also included in the glycan analysis was purified plasma derived Hemopexin obtained commercially (Athens Research Technologies) as a control. Glycan analysis included MALDI analysis to identify neutral and charged N-glycan structures, 2AA analysis to identify % neutral glycans, and in some instances total sialic acid analysis to determine total sialic acid content (See FIG. 3, further details in Example 2).

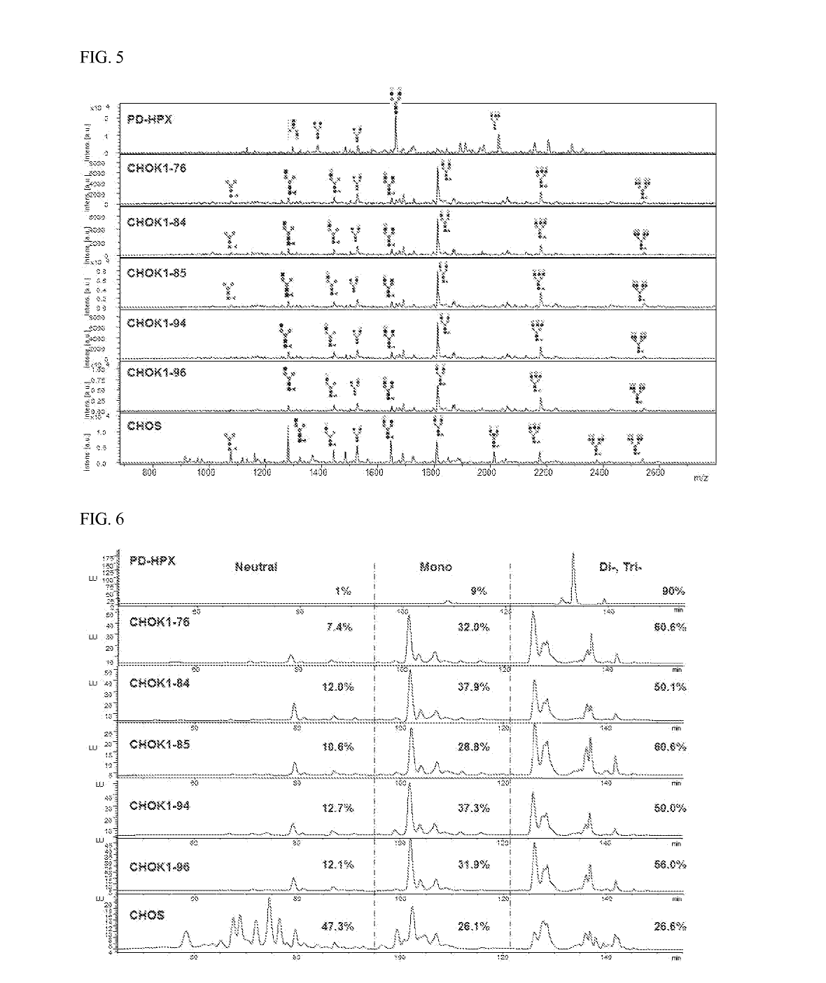

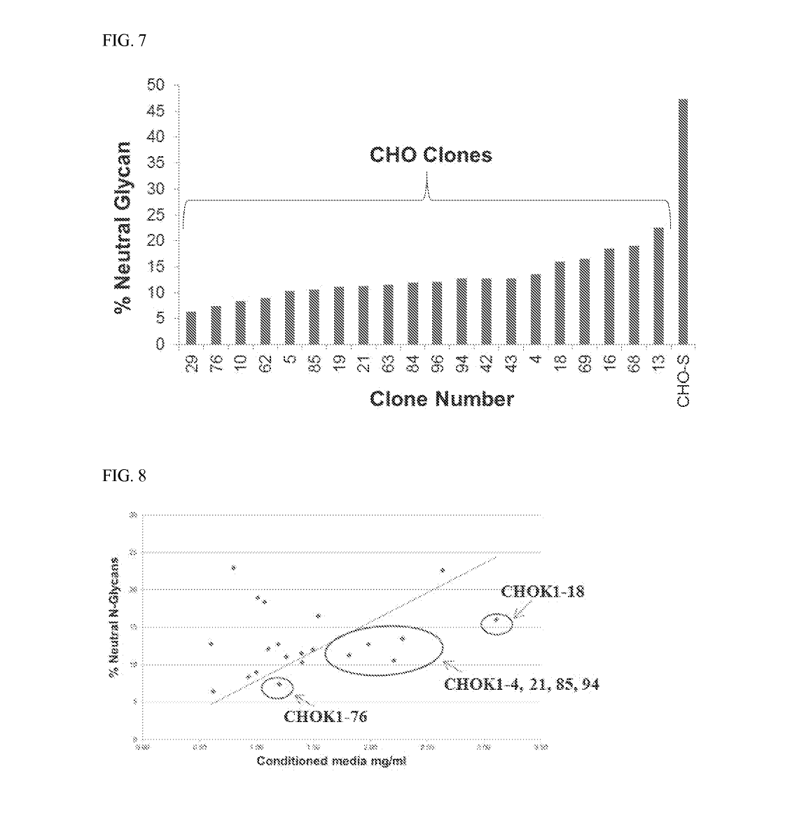

Unexpectedly, the Maldi and 2AA glycan analysis for Hemopexin purified from the CHO-S and 21 CHOK1 clones revealed significant differences in the glycan profiles (See FIGS. 4, 5, and 6). The MALDI sialylated N-glycan analysis revealed a more diverse pattern of glycans for the CHO-S derived Hemopexin compared to the CHOK1 clones. Furthermore, the percent neutral glycans present in the CHO-S derived material (47.8%) was significantly increased compared to that obtained with the CHOK1 derived material (6.3% to 24.6%). A reduced percent neutral glycan is inversely proportional to the degree of sialylation. Therefore, the material from the CHOK1 clones had a greater degree of sialylation based on this analysis. The % neutral glycans for all clones is shown in the graph in FIG. 7. Clones were selected for additional evaluation based on the percent neutral N-glycans and expression level (FIG. 8).

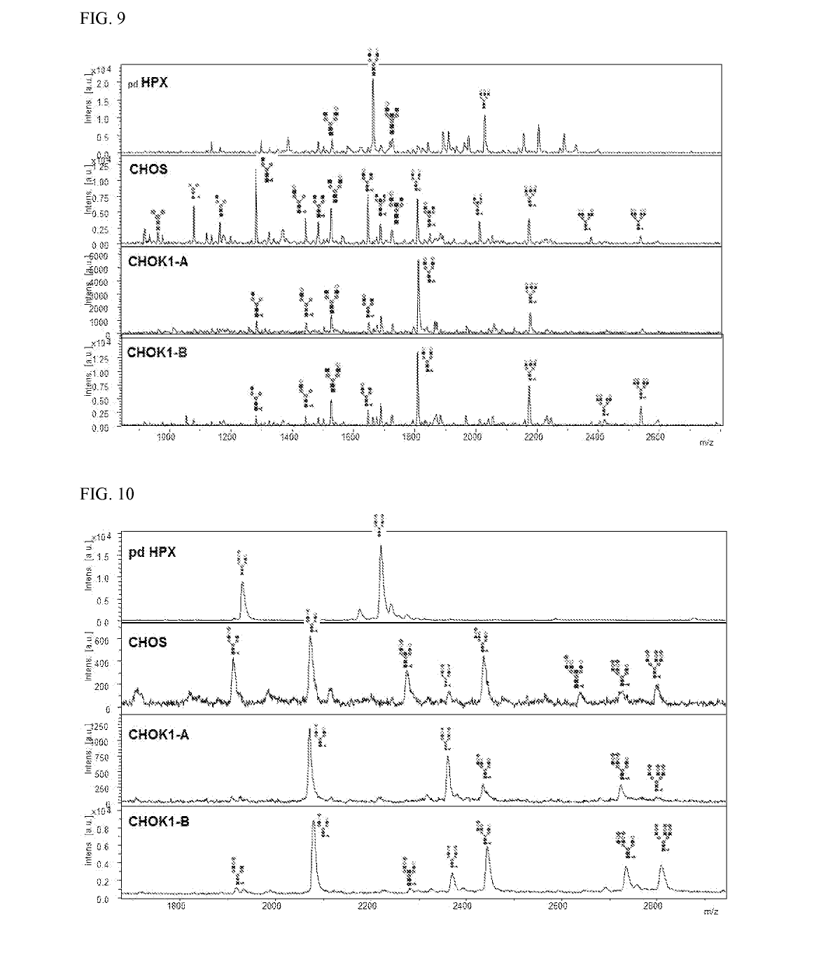

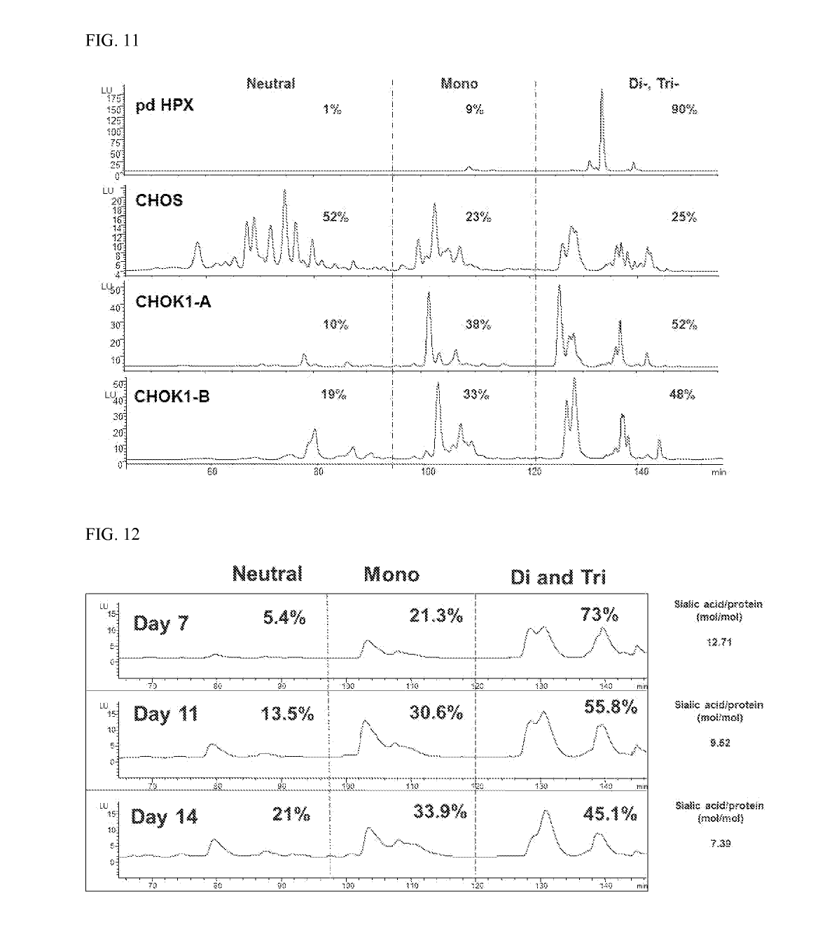

The CHO-S clone and CHOK1 clone 76 were used to produce material for a pharmacokinetic study. The MALDI analysis showing the neutral and charged N-glycans for these two preps (and Athens Research Plasma derived) is shown in FIGS. 9 and 10. The % neutral glycan data from 2AA analysis is shown in FIG. 11. The CHO-S produced material had 52% neutral glycan and the CHOK1-76 (batch A) produced material had 10% neutral glycan. A second batch of Hemopexin was purified from clone CHOK1-76 conditioned media that had an increased level of percent neutral glycans (19%) based on 2AA analysis. A table summarizing the percent neutral glycans for the four recombinantly produced preparations and the commercially obtaining plasma derived Hemopexin is shown below in Table 1. Preparations with lower percent total neutral N-glycans also have a higher level of fully sialylated N-glycans and a reduced level of N-glycans containing two or more uncharged terminal galactose moieties.

TABLE 1
Summary of % neutral glycan (2AA) and Maldi charged N-Glycan
analysis for preparations used in pharmacokinetic analysis.

| Hemopexin | Fully | One | Two | % Neutral |
|---|---|---|---|---|
| Batch | Sialylated | uncharged | Uncharged | Glycan |
| Plasma Derived | 67% | 33% | 0% | 0% |
| CHO-S derived | 11% | 64% | 25% | 52% |
| CHOK1 derived A | 32% | 58% | 10% | 10% |
| CHOK1 derived B | 22% | 55% | 22% | 19% |

Proteins from these preparations were evaluated in the pharmacokinetic analysis shown below.

Further analysis of CHOK1 clone 76 revealed that the level of percent neutral glycans present in protein purified using conditioned media from bioreactor cultures was dependent on the length of time the culture was carried. We inoculated a 10 L bioreactor (ActiCHOP media) with clone 76 obtained conditioned media at days 7, 11, and 14. The Hemopexin was purified from media collected on these days and then evaluated for % neutral glycan. The data (FIG. 12) revealed that there was a time dependent increase in the % neutral glycan during the bioreactor run. This can be due to either consumption of critical media components or the presence of sialidases in the media that lead to the removal of sialic acid over time. Conditions can be further optimized to reduce that % neutral glycans in the product using a combination of modified growth conditions, addition of media components in the feed, or addition of sialidase inhibitors into the media. Further one can also imagine certain known methods or techniques for adding various genes to these high producing cells that can enhance sialylation. This can include but not be limited to sialyltransferases and CMP-sialic acid transporters.

### EXAMPLE 2

### Glycan Analysis

2AA analysis—Neutral and sialylated N-glycans were analyzed by HPLC after labelling with fluorescent probe 2-aminobenzoic acid. N-glycans were released by using Glycosidase F (Oxford Glycosystem) followed by labelling with 2-aminobenzoic acid. The labelled samples were analyzed on NH2P40-2D column using 2% Acetic acid/1% Tetrahydrofuran in acetonitrile as solvent A and 5% acetic acid/1% Tetrahydrofuran /3% triethylamine in water as solvent B with fluorescence detection (Excitation 360 nm, Emission 425 nm).

MALDI analysis—For determining the structure of the glycans, N-glycans were released by using Glycosidase F, followed by MALDI-MS analysis. For neutral glycan analysis, 2,5-dihydroxybenzoic acid was used as a matrix while 2′,4′,6′-Trihydroxyacetophenone monohydrate was used for sialylated glycans analysis. For neutral N-glycan analysis, the data acquisition parameters were as follows: Ion Source 1: 20 kV, Ion source 2: 17kv, lens 9kv, reflector 1: 26, reflector 2: 14. For sialylated N-glycan analysis, the data acquisition parameters were as follows: Ion source 1: 20 kv, Ion source 2: 19kv, lens 5kv.

### EXAMPLE 3

### PK Study

The pharmacokinetic and disposition profile for recombinant (CHO-S and CHOK1 derived) and plasma derived (Athens Research Technologies) Hemopexins were evaluated in conscious, male Sprague-Dawley rats. The recombinant CHO-K1 derived Hemopexins were glycan-modified to reduce the percentage of neutral glycans. The recombinant CHO-S derived Hemopexin was not glycan-modified. Hemopexin was administered as a single intravenous dose at 3 mg/kg into the femoral vein. This study was performed using a Culex™ Automated Blood Sampling System (Bioanalytical Systems, Inc., Lafayette, Ind.). Following dosing, blood samples were collected serially through the jugular vein into collection tubes containing 5% sodium citrate as anticoagulant at pre-designated time points up to 72 hours. Subsequently, plasma was obtained from these samples and stored at −80° C. until analysis. Plasma levels of human Hemopexin were determined using a sandwich ELISA assay method with anti-human-Hemopexin antibody as capture and HRP-anti-human-Hemopexin antibody as detection to measure the total human Hemopexin in rat plasma.

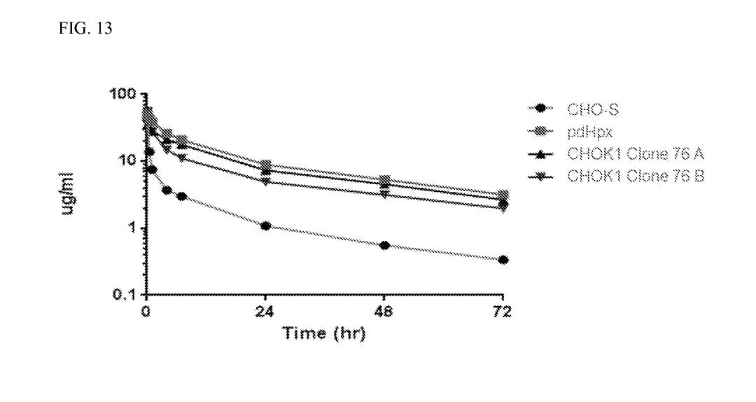

There was a clear correlation between percent neutral glycan in the batches and the clearance properties determined in the rat pharmacokinetic analysis (See FIG. 13). Hemopexin preparations with increased neutral glycan (reduced sialylation) had faster alpha phase and clearance rates, increased volumes of distribution, and reduced AUC (FIG. 13 and Table 2). This is presumably due to the more rapid clearance of insufficiently sialylated molecules through the asialylglycoprotein receptor. Clearance of improperly sialylated material through the asialylglycoprotein receptor can lead to the rapid clearance of free Hemopexin from the circulation before it scavenges free heme. Hemopexin molecules with improved sialylation can be cleared much more slowly until they bind heme. Upon binding heme the affinity for the LRP receptor is increased leading to removal of the Hemopexin-heme complex from circulation. By reducing the clearance through the asialylglycoprotein receptor the in vivo potency of the Hemopexin can be improved.

TABLE 2
Pharmacokinetic parameters for recombinant and plasma derived
human Hemopexin in Sprague-Dawley rats.

| | | CHOK1 | CHOK1 | |
|---|---|---|---|---|
| | CHOS | Clone 76 A | Clone 76 B | pdHX |
| AUCnorm (kg · h/L) | 40 | 142 | 237 | 260 |
| Cl (mL/h/kg) | 25 | 5.7 | 4.2 | 3.4 |
| Vss (mL/kg) | 690 | 230 | 170 | 130 |
| T½ (h) | 28 | 36 | 33 | 33 |

While the present embodiments have been described with reference to the specific embodiments and examples, it should be understood that various modifications and changes can be made and equivalents can be substituted without departing from the true spirit and scope of the claims appended hereto. The specification and examples are, accordingly, to be regarded in an illustrative rather than in a restrictive sense. Furthermore, the disclosure of all articles, books, patent applications and patents referred to herein are incorporated herein by reference in their entireties.

We claim:

1. A recombinant Hemopexin molecule for therapeutic treatment comprising a percentage of neutral glycans to total glycans in a range of from about 2 to about 30 percent as measured by HPLC after labelling with fluorescent probe 2-aminobenzoic acid.

2. A recombinant Hemopexin molecule as recited in claim 1, expressed from a CHO cell.

3. A recombinant Hemopexin as recited in claim 1, wherein the CHO cell comprises a CHO-K1 cell.

4. A recombinant Hemopexin molecule as recited in claim 1, wherein the recombinant Hemopexin molecule comprises a mammalian Hemopexin molecule.

5. A recombinant Hemopexin molecule for therapeutic treatment comprising a percentage of neutral glycans in a range of from about 2 to about 30 percent, a percentage of mono-sialylated glycans in a range of from about 2 to about 40 percent, and a percentage of di/tri sialylated glycans in a range of from about 20 to about 90 percent, as measured by HPLC after labelling with fluorescent probe 2-aminobenzoic acid.

6. The recombinant Hemopexin molecule recited in claim 5, wherein the Hemopexin molecule is used to treat the toxic effects of heme in a disease.

7. The recombinant Hemopexin molecule recited in claim 6, wherein the disease comprises sickle cell disease.

8. The recombinant Hemopexin molecule recited in claim 6, wherein the disease comprises β-thalassemia.

9. A method of making a recombinant Hemopexin molecule having a percent neutral glycan to total glycans in a range of from about 2 to about 30 percent as measured by HPLC after labelling with fluorescent probe 2-aminobenzoic acid, comprising:
(a) inserting a nucleic acid comprising a recombinant Hemopexin nucleic acid sequence into a CHO cell; and
(b) expressing the recombinant Hemopexin molecule from the CHO cell
wherein the percent neutral glycan of the recombinant Hemopexin is in a range of from about 2 to about 30 percent as measured by HPLC after labelling with fluorescent probe 2-aminobenzoic acid.

10. A method of making a recombinant Hemopexin molecule as recited in claim 9, wherein the CHO cell comprises a CHO-K1 cell.

11. A recombinant Hemopexin molecule for therapeutic treatment as recited in claim 1, where the percentage of neutral glycans to total glycans is less than 30 percent as measured by HPLC after labelling with fluorescent probe 2-aminobenzoic acid.

12. A recombinant Hemopexin molecule for therapeutic treatment as recited in claim 1, where the percentage of neutral glycans to total glycans is less than 20 percent as measured by HPLC after labelling with fluorescent probe 2-aminobenzoic acid.

13. A recombinant Hemopexin molecule for therapeutic treatment as recited in claim 1, where the percentage of neutral glycans to total glycans is less than 10 percent as measured by HPLC after labelling with fluorescent probe 2-aminobenzoic acid.

14. A method of therapeutic treatment comprising administering to a subject a recombinant Hemopexin molecule having a percentage neutral glycan to total glycans in a range of from about 2 to about 30 percent as measured by HPLC after labelling with fluorescent probe 2-aminobenzoic acid.

15. A method as recited in claim 14, wherein the recombinant Hemopexin molecule circulates in the blood stream at a sufficient half-life to bind free heme.

16. A recombinant Hemopexin molecule having a 90% or greater homology to SEQ ID NO: 1, wherein the percentage of neutral glycans to total glycans is in a range of from about 2 to about 30 percent as measured by HPLC after labelling with fluorescent probe 2-aminobenzoic acid.

17. A recombinant Hemopexin molecule as recited in claim 1 or 16, wherein the molecule is used for treating a disease selected from the group consisting of sickle cell disease, β-thalassemia, ischemia reperfusion, erythropoeitic protoporphyria, porphyria cutanea tarda, malaria, rheumatoid arthritis, anemia associated with inflammation, hemochromatosis, paroxysmal nocturnal hemoglobinuria (PNH), glucose-6-phosphate dehydrogenase deficiency, hemolytic uremic syndrome (HUS), thrombotic thrombocytopenic purpura (TTP), pre-eclampsia, sepsis, acute bleeding, and complications associated with transfusion with blood or blood substitutes, and organ preservation associated with transplantation.

18. A method for exporting heme from a cell comprising contacting the cell with a recombinant Hemopexin molecule as recited in claim 1 or 16.

19. A method of treating a disorder associated with free heme toxicity comprising administering to a subject in need thereof a therapeutically effective amount of a recombinant Hemopexin molecule as recited in claim 1 or 16.

20. The method of claim 19, wherein the disorder is selected from sickle cell disease, β-thalessemia, erythropoeitic protoporphyria, porphyria cutanea tarda, ischemia reperfusion, and malaria.

21. A method of treating a disorder associated with excess intracellular heme comprising administering to a subject in need thereof a therapeutically effective amount of a recombinant Hemopexin molecule as recited in claim 1 or 16.

22. The method of claim 21, wherein the disorder is selected from rheumatoid arthritis, anemia associated with inflammation, and conditions in which iron accumulates in macrophage cells.


SEQUENCE LISTING (4 sequences)

SEQ ID NO: 1 - protein, 462 aa, Homo sapiens
MARVLGAPVALGLWSLCWSLAIATPLPPTSAHGNVAEGETKPDPDVTERCSDGWSFDATTLDDNGTMLFFKGEFVWKSHKWDRELISERWKNFPSPVDAAFRQGHNSVFLIKGDKVWVYPPEKKEKGYPKLLQDEFPGIPSPLDAAVECHRGECQAEGVLFFQGDREWFWDLATGTMKERSWPAVGNCSSALRWLGRYYCFQGNQFLRFDPVRGEVPPRYPRDVRDYFMPCPGRGHGHRNGTGHGNSTHHGPEYMRCSPHLVLSALTSDNHGATYAFSGTHYWRLDTSRDGWHSWPIAHQWPQGPSAVDAAFSWEEKLYLVQGTQVYVFLTKGGYTLVSGYPKRLEKEVGTPHGIILDSVDAAFICPGSSRLHIMAGRRLWWLDLKSGAQATWTELPWPHEKVDGALCMEKSLGPNSCSANGPGLYLIHGPNLYCYSDVEKLNAAKALPQPQNVTSLLGCTH

SEQ ID NO: 2 - DNA, 1410 bp, Homo sapiens
atggctagggtactgggagcacccgttgcactggggttgtggagcctatgctggtctctggccattgccacccctcttcctccgactagtgcccatgggaatgttgctgaaggcgagaccaagccagacccagacgtgactgaacgctgctcagatggctggagctttgatgctaccaccctggatgacaatggaaccatgctgttttttaaaggggagtttgtgtggaagagtcacaaatgggaccgggagttaatctcagagagatggaagaatttccccagccctgtggatgctgcattccgtcaaggtcacaacagtgtctttctgatcaagggggacaaagtctgggtataccctcctgaaaagaaggagaaaggatacccaaagttgctccaagatgaatttcctggaatcccatccccactggatgcagctgtggaatgtcaccgtggagaatgtcaagctgaaggcgtcctcttcttccaaggtgaccgcgagtggttctgggacttggctacgggaaccatgaaggagcgttcctggccagctgttgggaactgctcctctgccctgagatggctgggccgctactactgcttccagggtaaccaattcctgcgcttcgaccctgtcaggggagaggtgcctcccaggtacccgcgggatgtccgagactacttcatgccctgccctggcagaggccatggacacaggaatgggactggccatgggaacagtacccaccatggccctgagtatatgcgctgtagcccacatctagtcttgtctgcactgacgtctgacaaccatggtgccacctatgccttcagtgggacccactactggcgtctggacaccagccgggatggctggcatagctggcccattgctcatcagtggccccagggtccttcagcagtggatgctgccttttcctgggaagaaaaactctatctggtccagggcacccaggtatatgtcttcctgacaaagggaggctataccctagtaagcggttatccgaagcggctggagaaggaagtcgggacccctcatgggattatcctggactctgtggatgcggcctttatctgccctgggtcttctcggctccatatcatggcaggacggcggctgtggtggctggacctgaagtcaggagcccaagccacgtggacagagcttccttggccccatgagaaggtagacggagccttgtgtatggaaaagtcccttggccctaactcatgttccgccaatggtcccggcttgtacctcatccatggtcccaatttgtactgctacagtgatgtggagaaactgaatgcagccaaggcccttccgcaaccccagaatgtgaccagtctcctgggctgcactcaccaccatcaccaccatcatcaccat

SEQ ID NO: 3 - DNA, 1451 bp, Homo sapiens
accggtgaattcgccgccaccatggctcgcgttcttggtgcccctgttgccctcggtctttggtccctctgttggtcacttgctattgccactccgctgcctccgaccagcgcgcacggaaatgtggccgaaggcgaaactaagccagaccctgacgtgaccgagagatgcagcgacggatggagcttcgacgctactaccctggatgataacggcactatgctgttctttaagggggagttcgtgtggaagtcgcataagtgggaccgggagctcatctcagaaaggtggaagaactttccgtccccggtcgacgctgcatttcggcagggacacaattccgtgttcctgatcaagggggacaaagtgtgggtgtacccacctgagaaaaaggagaaaggttacccaaagctgctccaagatgagttcccgggcatcccctcgcccctcgacgcggcagtggaatgccatagaggcgaatgccaagcagaaggcgtgctgtttttccaaggggacagagaatggttctgggacctggctacgggaaccatgaaggaacgctcctggccagccgtgggaaattgctccagcgcactgcgatggctgggaagatactactgtttccaaggaaatcagtttcttcgcttcgatcctgtccgcggagaggtgcccccacggtacccgcgggacgtgcgcgactattttatgccgtgtccgggacggggccatggccaccggaacggaaccgggcatggaaactcgactcatcacggacctgagtacatgaggtgcagcccgcatctcgtgctgtccgccctcacctccgacaaccatggggctacctatgcattctcgggtactcactactggaggctggatacctcacgggatggatggcactcgtggccgatcgcgcaccagtggccacagggcccctcagcagtcgatgccgctttctcatgggaggaaaagctctacctggtgcagggtacccaagtctacgtgttcctcactaagggaggctacacgctcgtgtcgggctacccaaagagactggagaaggaggtggggactccccatggaatcatcctggactcggtcgatgctgcattcatctgcccgggaagctcgcggctgcacattatggcgggacgccgcctttggtggttggacttgaaatccggcgcccaggcgacttggactgaacttccgtggcctcacgagaaggtcgacggagcgttgtgcatggaaaaatctctgggaccaaactcctgcagcgccaacggaccgggattgtacctgatccacggaccgaatctgtactgctactcggatgtcgaaaaattgaacgcggccaaggcgctccctcagccgcagaacgtgacctcgctgcttggatgtacacaccaccaccatcaccatcatcaccaccattaggcggccgcgctagc

SEQ ID NO: 4 - protein, 462 aa, Homo sapiens; identical to SEQ ID NO: 1